(19) United States
(12) Patent Application Publication
(10) Pub. No.: US 2023/0000545 A1
(43) Pub. Date: Jan. 5, 2023

(54) ABLATION CATHETER TIP WITH FLEXIBLE ELECTRONIC CIRCUITRY

(71) Applicant: St. Jude Medical, Cardiology Division, Inc., St. Paul, MN (US)

(72) Inventor: Brett Hillukka, St. Michael, MN (US)

(21) Appl. No.: 17/779,832

(22) PCT Filed: Nov. 25, 2020
(86) PCT No.: PCT/IB2020/061157

§ 371 Date: May 25, 2022

Related U.S. Application Data
(60) Provisional application No. 62/940,737, filed on Nov. 26, 2019.

(51) Int. Cl. A61B 18/14

(52) U.S. Cl. CPC A61B 18/1492; A61B 2018/00577

(57) ABSTRACT

Aspects of the present disclosure are directed to, for example, a high-thermal-sensitivity ablation catheter tip. More specifically, various aspects of the present disclosure are directed to improved thermocouple response to temperature changes associated with an ablation electrode and/or tissue in contact therewith. Such an ablation catheter tip facilitates reduced lag in an ablation control system's response to the sensed temperature changes.

### CROSS-REFERENCE TO RELATED APPLICATIONS

This application claims the benefit of U.S. provisional application No. 62/940,737, filed on 26 Nov. 2019, which is hereby incorporated by reference as though fully set forth herein.

This application incorporates by reference as though fully set forth herein: U.S. application Ser. No. 15/088,036, filed 31 Mar. 2016, now pending, which claims the benefit of U.S. provisional application No. 62/141,066, filed 31 Mar. 2015; U.S. application Ser. No. 15/088,052, filed 31 Mar. 2016, now pending, which claims the benefit of U.S. provisional application No. 62/198,114, filed 28 Jul. 2015; U.S. application Ser. No. 15/723,701, filed 3 Oct. 2017, now pending, which claims the benefit of U.S. provisional application No. 62/404,038, filed 4 Oct. 2016; U.S. application Ser. No. 15/724,157, filed 3 Oct. 2017, now pending, which claims the benefit of U.S. provisional application No. 62/404,060, filed 4 Oct. 2016; international application no. PCT/US2017/049264, filed 30 Aug. 2017, now pending, which claims the benefit of U.S. provisional application No. 62/404,013, filed 4 Oct. 2016; U.S. provisional application No. 62/642,178, filed 13 Mar. 2018; U.S. provisional application No. 62/824,840, filed 27 Mar. 2019; U.S. provisional application No. 62/824,844, filed 27 Mar. 2019; U.S. provisional application No. 62/824,846, filed 27 Mar. 2019; U.S. provisional application No. 62/832,246, filed 10 Apr. 2019; U.S. provisional application No. 62/832,248, filed 10 Apr. 2019; and United States provisional application concurrently filed herewith under docket no. 13675USL1/065513-002153.

### BACKGROUND OF THE DISCLOSURE

### a. Field

The instant disclosure relates to various types of medical catheters, in particular catheters for diagnostics within, and/or treatment of, a patient's cardiovascular system. In one embodiment, the instant disclosure relates to an ablation catheter for treating cardiac arrhythmias within a cardiac muscle. Various aspects of the instant disclosure relate to force sensing systems capable of determining a force applied at a distal tip of the ablation catheter.

The present disclosure further relates to low thermal mass ablation catheter tips (also known as high-thermal-sensitivity catheter tips) and to systems for controlling the delivery of radio-frequency energy to such catheter tips during ablation procedures.

### b. Background

Exploration and treatment of various organs or vessels has been made possible using catheter-based diagnostic and treatment systems. These catheters may be introduced through a vessel leading to the cavity of the organ to be explored, and/or treated. Alternatively, the catheter may be introduced directly through an incision made in the wall of the organ. In this manner, the patient avoids the trauma and extended recuperation times typically associated with open surgical procedures.

The human heart routinely experiences electrical currents traversing its many layers of tissue. Just prior to each heart contraction, the heart depolarizes and repolarizes as electrical currents spread across the heart. In healthy hearts, the heart will experience an orderly progression of depolarization waves. In unhealthy hearts, such as those experiencing atrial arrhythmia, including for example, ectopic atrial tachycardia, atrial fibrillation, and atrial flutter, the progression of the depolarization wave becomes chaotic.

Catheters are used in a variety of diagnostic and/or therapeutic medical procedures to diagnose and correct conditions such as atrial arrhythmia. Typically, in such a procedure, a catheter is manipulated through a patient's vasculature to the patient's heart carrying one or more end effectors which may be used for mapping, ablation, diagnosis, or other treatment. Where an ablation therapy is desired to alleviate symptoms including atrial arrhythmia, an ablation catheter imparts ablative energy to cardiac tissue to create a lesion in the cardiac tissue. The lesioned tissue is less capable of conducting electrical signals, thereby disrupting undesirable electrical pathways and limiting or preventing stray electrical signals that lead to arrhythmias. The ablation catheter may utilize ablative energy including, for example, radio frequency (RF), cryoablation, laser, chemical, and high-intensity focused ultrasound. Ablation therapies often require precise positioning of the ablation catheter, as well as precise pressure exertion for optimal ablative-energy transfer into the targeted myocardial tissue. Excess pressure between the ablation catheter tip and the targeted myocardial tissue may result in excessive ablation which may permanently damage the cardiac muscle and/or surrounding nerves. When the contact pressure between the ablation catheter tip and the targeted myocardial tissue is below a target pressure, the efficacy of the ablation therapy may be reduced.

Ablation therapies are often delivered by making a number of individual ablations in a controlled fashion in order to form a lesion line. To improve conformity of the individual ablations along the lesion line, it is desirable to precisely control the position at which the individual ablations are conducted, the ablation period, the ablation electrode and/or target tissue temperature, and the contact pressure between the ablation catheter tip and the targeted tissue. All of these factors affect the conformity of the resulting lesion line.

The foregoing discussion is intended only to illustrate the present field and should not be taken as a disavowal of claim scope.

### BRIEF SUMMARY OF THE DISCLOSURE

It is desirable to precisely control the delivery of RF energy to an ablation catheter to enable the creation of uniform lesions along a lesion line, and mitigate against overheating of tissue. Accordingly, aspects of the present disclosure are directed toward an ablation catheter tip including high thermal sensitivity materials which facilitate near real-time temperature sensing at the ablation catheter tip. Further aspects of the present disclosure are directed to electrophysiology signal detection and mapping on, or in close proximity to, the ablation catheter tip.

Aspects of the present disclosure are directed to a high-thermal-sensitivity ablation catheter tip. In one such embodiment the high-thermal-sensitivity ablation catheter tip includes an ablation electrode and a flexible electronic circuit. The ablation electrode delivers an ablation therapy to tissue in contact with or in close proximity thereto. The flexible electronic circuit is coupled to an external surface of the ablation electrode, and includes one or more electrodes that sense electrophysiology characteristics of the tissue in contact with or in close proximity to the ablation electrode. In more specific embodiments, the ablation electrode may include a trench extending into the external surface. The trench positions and couples the flexible electronic circuit thereto, and positions a sensing surface of the one or more electrodes flush with the external surface of the ablation electrode.

Another embodiment of the present disclosure is also directed to a high-thermal-sensitivity ablation catheter tip. In such an embodiment the tip includes an ablation electrode, a distal flexible electronic circuit, and a proximal flexible electronic circuit. The ablation electrode delivers an ablation therapy to tissue in contact with or in close proximity thereto. The ablation electrode includes a conductive tip, a conductive shell, and a transition region between the conductive tip and the conductive shell. The distal flexible electronic circuit includes a first plurality of electrodes which extend through apertures in a transition region of the ablation electrode. The proximal flexible electronic circuit includes a second plurality of electrodes which extend through apertures at a proximal end of the conductive shell. In more specific embodiments, the distal flexible electronic circuit further includes an additional electrode extending through the conductive tip and positioned along a longitudinal axis of the catheter tip.

The foregoing and other aspects, features, details, utilities, and advantages of the present disclosure will be apparent from reading the following description and claims, and from reviewing the accompanying drawings.

### DESCRIPTION OF THE DRAWINGS

Various example embodiments may be more completely understood in consideration of the following detailed description in connection with the accompanying drawings.

FIG. 1 is a diagrammatic overview of an ablation catheter system including a force sensing subsystem, consistent with various embodiments of the present disclosure;

FIG. 2A is a side view of an ablation catheter tip assembly, consistent with various embodiments of the present disclosure;

FIG. 2B is a perspective side view of the ablation catheter tip assembly of FIG. 2A, consistent with various embodiments of the present disclosure;

FIG. 2C is a front view of the ablation catheter tip assembly of FIG. 2A, consistent with various embodiments of the present disclosure;

FIG. 2D is a perspective side view of a partial ablation catheter tip assembly of FIG. 2A, consistent with various embodiments of the present disclosure;

FIG. 2E is a cross-sectional side view of a proximal portion of the ablation catheter tip assembly of FIG. 2A, consistent with various embodiments of the present disclosure;

FIG. 3A is a perspective side view of an ablation catheter tip assembly, consistent with various embodiments of the present disclosure;

FIG. 3B is a front view of the ablation catheter tip assembly of FIG. 3A, consistent with various embodiments of the present disclosure;

FIG. 3C is a side view of the ablation catheter tip assembly of FIG. 3A, consistent with various embodiments of the present disclosure;

FIG. 4A is a perspective side view of a conductive tip shell, consistent with various embodiments of the present disclosure;

FIG. 4B is a back view of the conductive tip shell of FIG. 4A, consistent with various embodiments of the present disclosure;

FIG. 4C is a perspective back view of the conductive tip shell of FIG. 4A, consistent with various embodiments of the present disclosure;

FIG. 5A is a front view of an ablation catheter tip assembly, consistent with various embodiments of the present disclosure;

FIG. 5B is a side view of the ablation catheter tip assembly of FIG. 5A, consistent with various embodiments of the present disclosure;

FIG. 5C is a perspective side view of the ablation catheter tip assembly of FIG. 5A, consistent with various embodiments of the present disclosure;

FIG. 6 is a perspective side view of an ablation catheter tip assembly, consistent with various embodiments of the present disclosure. A conductive shell of the ablation catheter tip assembly is illustrated in transparency to facilitate discussion of the internal features of the assembly.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION OF EMBODIMENTS

Various aspects of the present disclosure are directed to medical catheters, in particular, catheters for diagnostics within, and/or treatment of, a patient's cardiovascular system. In many embodiments, the instant disclosure relates to an ablation catheter for treating cardiac arrhythmias within a cardiac muscle, such as atrial fibrillation. To diagnose and treat symptoms related to atrial fibrillation, for example, a distal tip of the catheter may include electrodes (also referred to as sensing electrodes and/or electrophysiology electrodes). The electrodes may also be used to confirm a successful ablation therapy by verifying isolation of stray electrical signals (e.g., those caused by arrhythmic foci) from a left atrium of the cardiac muscle, for example.

During a non-invasive intravascular ablation therapy, it is desirable to precisely control delivery of energy (e.g., radio-frequency, thermal, etc.) at a distal tip of the catheter to enable proper lesion formation in the myocardial tissue. During ablation therapy, generator power must remain sufficiently high to form adequate lesions, while mitigating against overheating of tissue (associated with steam pops, charring and/or coagulation on the ablation catheter tip). Accordingly, aspects of the present disclosure are directed toward an ablation catheter tip with high thermal sensitivity materials which facilitate near real-time temperature sensing at the ablation catheter tip.

Various embodiments of the present disclosure are directed to an ablation catheter with thermocouples and/or electrodes positioned at a distal end of an ablation tip (and/or a second set of thermocouples and/or electrodes positioned at a proximal end of the ablation tip).

To improve safety and effectiveness of intracardiac ablation therapies, aspects of the present disclosure monitor tip-tissue temperatures and local electrograms. The need to monitor tip-tissue temperature has become more critical as many physicians have moved to therapy protocols utilizing higher power and shorter duration ablations. The ablation electrode is commonly manufactured with sharp corners at a proximal end, and therefore during ablation therapy may be subject to “hot zones” due to the increased current density. These “hot zones,” when contacted by myocardial tissue may subject the tissue to undesirably high temperatures which can, in extreme cases, result in steam pops. Aspects of the present disclosure monitor the temperature at or near the proximal end of the ablation electrode during ablation therapy, and in response to temperature signals received by controller circuitry, the controller circuitry may adjust power delivery to the ablation electrode—thereby improving therapy efficacy, and safety (e.g., by reducing the possibility of char, coagulum, or a steam pop).

Aspects of the present disclosure are further directed to increasing ablation catheter capability. For example, it has been discovered that ablation catheters may benefit from the ability to measure localized temperature, electrograms, voltage, and impedance with the ablation catheter—improving patient safety and facilitating improved patient outcomes. Such capabilities, including electrodes and thermocouples may be implemented on, or in close proximity to, an ablation electrode. Various embodiments of the present disclosure facilitate capturing local electrophysiology properties and/or local temperature measurements at the ablation catheter tip, while further minimizing complexity and cost of the catheter.

To minimize complexity and cost of an ablation catheter, various embodiments of a flexible ablation catheter tip utilize one or more flex circuits including thermocouples and microelectrodes which are communicatively coupled to controller circuitry. These one or more flex circuit(s) may be routed through an internal lumen of the catheter shaft and extend proximally along a length of the catheter shaft toward controller circuitry which is communicatively coupled to a catheter handle of the ablation catheter via a connector.

Several embodiments of the present disclosure are directed to the integration of localized temperature and/or voltage/impedance information into an ablation catheter, with the safety profile of the FlexAbility™ ablation catheter (manufactured by Abbott Laboratories). In such an embodiment, a flexible electronic circuit (with thermocouples and/or electrodes communicatively coupled thereto) may be wrapped around and/or attached to an internal diameter of a conductive shell that operates as the ablation electrode. The electrodes may extend through apertures in the ablation electrode and be electrically insulated therefrom. The thermocouples may be placed into thermal contact with an inner diameter of the ablation electrode. In yet other embodiments, portions of the one or more flexible circuits may extend outside the conductive shell and be coupled to an external diameter of the conductive shell—facilitating direct tissue contact between the electrodes and target tissue. The use of the flexible electronic circuit assembly reduces assembly complexity by placing the flexible electronic circuit (or flex circuit) outside of an internal diameter of the tip, where space is more limited. Furthermore, external temperature sensor positioning may improve temperature sensing accuracy (as the thermocouples are not in contact with or otherwise washed out by catheter irrigation flow within the catheter shaft) and benefit from near real-time temperature sensing due to the thermal conductivity between the ablation electrode and flex circuit. The flex circuit may then communicate electrical signals indicative of the sensed temperatures at the one or more thermocouples to controller circuitry.

In one embodiment of the present disclosure, a flex circuit is integrated into an outer surface of an ablation electrode (also referred to as the conductive tip shell). One or more trenches extending into the outer surface of the ablation electrode facilitate flush coupling (or nearly flush) of electrodes on the flex circuit and the outer surface of the ablation electrode-facilitating a smooth, atraumatic transition between the tip, flex circuit, and the ablation electrode.

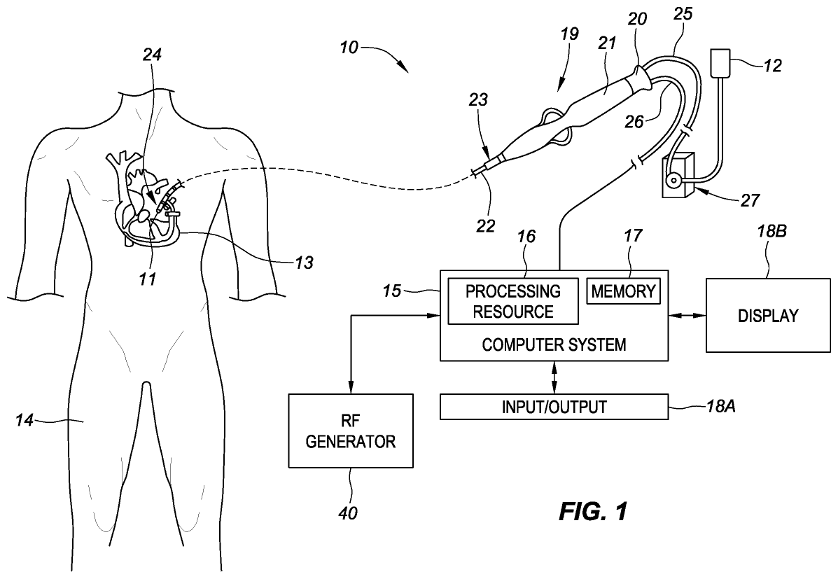

Referring now to the drawings wherein like reference numerals are used to identify identical components in the various views, FIG. 1 generally illustrates an ablation catheter system 10 having an elongated medical device 19 that includes a sensor assembly 11 (e.g., fiber optic based distance measurement sensor) at a distal end 24, and configured to be used in the body for medical procedures. The elongated medical device 19 may be used for diagnosis, visualization, and/or treatment of tissue 13 (such as cardiac or other tissue) in the body. For example, the medical device 19 may be used for ablation therapy of tissue 13 or mapping of a patient's body 14. FIG. 1 further illustrates various sub-systems included in the ablation catheter system 10. The system 10 may include a main computer system 15 (including an electronic control unit 16 and data storage 17, e.g., memory). The computer system 15 may further include conventional interface components, such as various user input/output mechanisms 18A and a display 18B, among other components. Information provided by the sensor assembly 11 may be processed by the computer system 15 and may provide data to the clinician via the input/output mechanisms 18A and/or the display 18B, or in other ways as described herein. Specifically, the display 18B may visually communicate a force exerted on the elongated medical device 19—where the force exerted on the elongated medical device 19 is detected in the form of a deformation of at least a portion of the elongated medical device by the sensor assembly 11, and the measured deformation is processed by the computer system 15 to determine the force exerted.

In the illustrative embodiment of FIG. 1, the elongated medical device 19 may include a cable connector or interface 20, a handle 21, a tubular body or shaft 22 having a proximal end 23 and a distal end 24. The elongated medical device 19 may also include other conventional components not illustrated herein, such as a temperature sensor, additional electrodes, and corresponding conductors or leads. The connector 20 may provide mechanical, fluid and/or electrical connections for cables 25, 26 extending from a fluid reservoir 12 and a pump 27 and the computer system 15, respectively. The connector 20 may comprise conventional components known in the art and, as shown, may be disposed at the proximal end of the elongated medical device 19.

The handle 21 provides a portion for a user to grasp or hold the elongated medical device 19 and may further provide a mechanism for steering or guiding the shaft 22 within the patient's body 14. For example, the handle 21 may include a mechanism configured to change the tension on a pull-wire extending through the elongated medical device 19 to the distal end 24 of the shaft 22 or some other mechanism to steer the shaft 22. The handle 21 may be conventional in the art, and it will be understood that the configuration of the handle 21 may vary. In an embodiment, the handle 21 may be configured to provide visual, auditory, tactile and/or other feedback to a user based on information received from the sensor assembly 11. For example, if contact to tissue 13 is made by distal end 24, the sensor assembly 11 may transmit data to the computer system 15 indicative of contact. In response to the computer system 15 determining that the data received from the sensor assembly 11 is indicative of contact between the distal end 24 and a patient's body 14, the computer system 15 may operate a light-emitting-diode on the handle 21, a tone generator, a vibrating mechanical transducer, and/or other indicator(s), the outputs of which could vary in proportion to the calculated contact force.

The computer system 15 may utilize software, hardware, firmware, and/or logic to perform a number of functions described herein. The computer system 15 may be a combination of hardware and instructions to share information. The hardware, for example may include processing resource 16 and/or a memory 17 (e.g., non-transitory computer-readable medium (CRM) database, etc.). A processing resource 16, as used herein, may include a number of processors capable of executing instructions stored by the memory resource 17. Processing resource 16 may be integrated in a single device or distributed across multiple devices. The instructions (e.g., computer-readable instructions (CRI)) may include instructions stored on the memory 17 and executable by the processing resource 16 for force detection.

The memory resource 17 is communicatively coupled with the processing resource 16. A memory 17, as used herein, may include a number of memory components capable of storing instructions that are executed by processing resource 16. Such a memory 17 may be a non-transitory computer readable storage medium, for example. The memory 17 may be integrated in a single device or distributed across multiple devices. Further, the memory 17 may be fully or partially integrated in the same device as the processing resource 16 or it may be separate but accessible to that device and the processing resource 16. Thus, it is noted that the computer system 15 may be implemented on a user device and/or a collection of user devices, on a mobile device and/or a collection of mobile devices, and/or on a combination of the user devices and the mobile devices.

The memory 17 may be communicatively coupled with the processing resource 16 via a communication link (e.g., path). The communication link may be local or remote to a computing device associated with the processing resource 16. Examples of a local communication link may include an electronic bus internal to a computing device where the memory 17 is one of a volatile, non-volatile, fixed, and/or removable storage medium in communication with the processing resource 16 via the electronic bus.

In various embodiments of the present disclosure, the computer system 15 may receive optical signals from a sensor assembly 11 via one or more optical fibers extending a length of the catheter shaft 22. A processing resource 16 of the computer system 15 may execute an algorithm stored in memory 17 to compute a force exerted on distal end 24, based on the received optical signals.

U.S. Pat. No. 8,567,265 discloses various optical force sensors for use in medical catheter applications, such optical force sensors are hereby incorporated by reference as though fully disclosed herein.

FIG. 1 further depicts an RF generator 40 operatively connected to the computer system 15, which is operatively connected to the elongated medical device 19. In this figure, a number of possible wired and/or wireless communication pathways are shown. For example, the computer system 15 may receive temperature feedback readings from at least one temperature sensor mounted on or near the distal end 24 of the catheter shaft 22. In various embodiments disclosed herein, the catheter may include multiple thermal sensors (for example, thermocouples or thermistors), as described further below. The temperature feedback readings may be the highest reading from among all of the individual temperature sensor readings, or it may be, for example, an average of all of the individual readings from all of the temperature sensors. The computer system 15 may then communicate to the RF generator 40 the highest temperature measured by any of the plurality of temperature sensors mounted within the sensor assembly 11. This could be used to trigger a temperature-based shutdown feature in the RF generator for patient safety. In other words, the temperature reading or readings from the catheter may be sent to the computer system 15, which may then feed the highest temperature reading to the RF generator 40 so that the generator can engage its safety features and shut down if the temperature reading exceeds a (safety) threshold.

In an alternative operation of the system 10 of FIG. 1, the computer system 15, in response to elevated temperature feedback from the thermal sensors, may operate the RF generator 40 in a pulsed manner. By pulsing the RF signal, the power can remain at a desired power level (e.g., 50 or 60 Watts) rather than being reduced to an ineffective level when excessive temperature is sensed by the catheter tip. In particular, rather than reducing the power to control temperature, the power may be delivered in a pulsed manner; by pulsing the RF signals, and controlling the length of pulses and gaps between pulses, tip temperature may be controlled as a surrogate for controlling actual tissue temperature. Similarly, instead of pulsing the power, the power may also be titrated in such a manner.

In the embodiment depicted in FIG. 1, the RF generator 40 may include pulse control hardware, software, and/or firmware built into the generator itself.

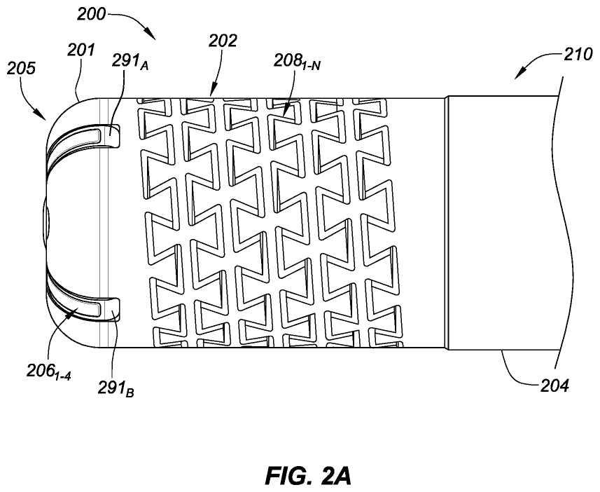

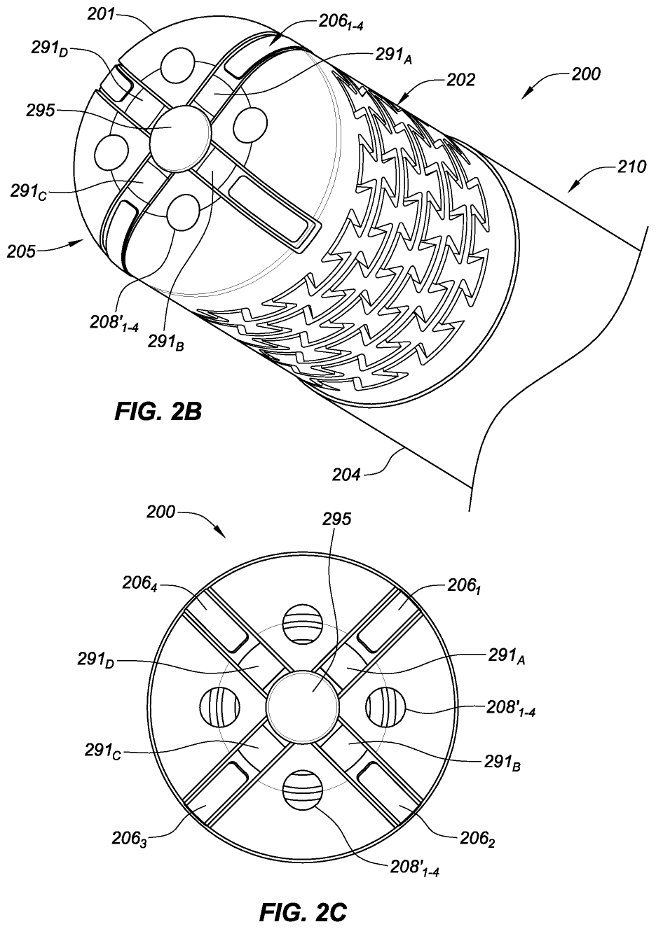

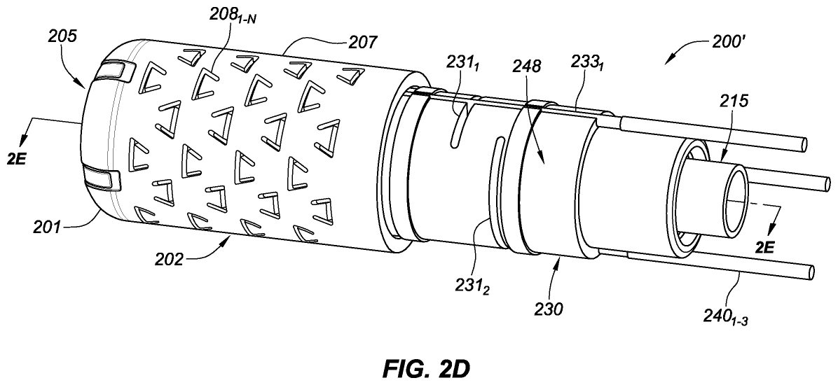

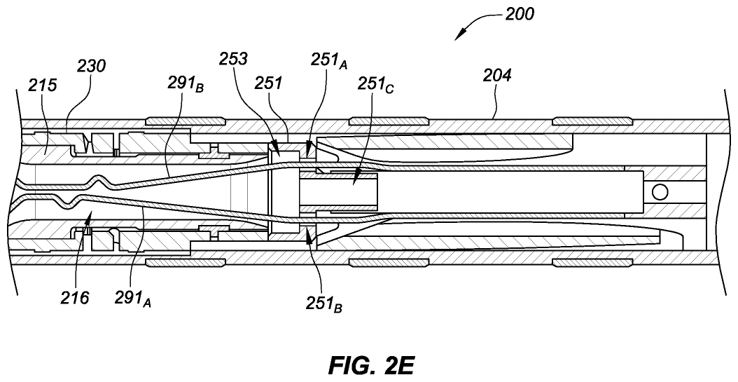

FIG. 2A is a side view of an ablation catheter tip assembly 200, FIG. 2B is a perspective side view of the ablation catheter tip assembly of FIG. 2A, and FIG. 2C is a front view of the ablation catheter tip assembly of FIG. 2A. The ablation catheter tip assembly 200 includes a conductive tip 201 and a conductive shell 202 coupled to a distal end of a catheter shaft 204. In the present embodiment, the conductive shell and conductive tip are capable of receiving a radio-frequency energy from a radio-frequency generator (e.g., RF Generator 40 as shown in FIG. 1), and transmitting that energy into myocardial tissue in contact with the conductive shell and/or conductive tip (or in close proximity thereto). In some embodiments (as shown in FIG. 2A), the ablation catheter is irrigated and includes a plurality of irrigant apertures 208_{1-N} which extend through the conductive shell 202. In further embodiments, distal irrigant apertures 208′₁₋₄ may also be utilized which extend through the conductive tip 201. The plurality of irrigant apertures deliver irrigant into contact with the targeted tissue for ablation therapy. The plurality of irrigant apertures 208_{1-N} may also facilitate desirable flexible characteristics of the ablation catheter tip assembly 200—such that in response to contact with target tissue the assembly deforms to increase a tip-tissue contact area.

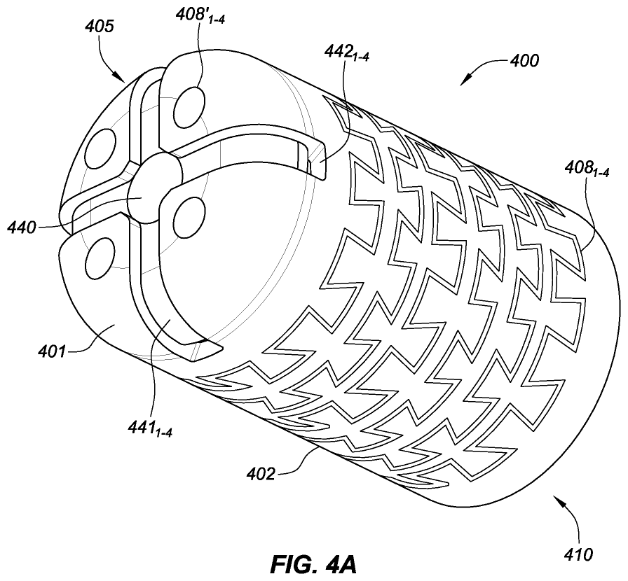

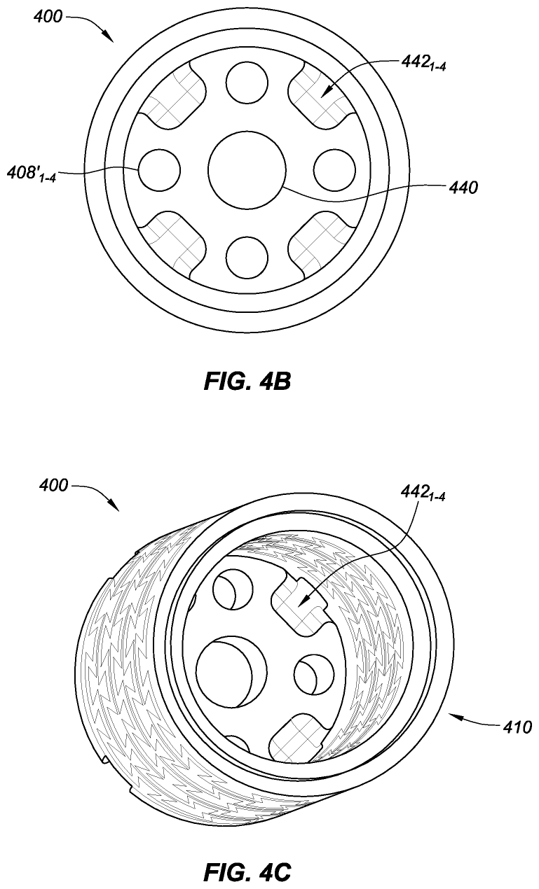

At a distal end 205 of the tip assembly 200, and in some embodiments at a juncture (or transition region) between the conductive shell 202 and the conductive tip 201, flexible circuits 291_{A-D} may extend from an interior cavity of the catheter and extend distally along a surface of the conductive tip. The flexible circuits 291_{A-D} may all be entirely separate flex circuits or be fingers off of the same flex circuit. The flexible circuits 291_{A-D} include electrodes 206₁₋₄ (or are communicatively coupled thereto) which facilitate the detection of electrophysiological characteristics of tissue in contact with conductive tip 201 of the tip assembly 200. For example, the flexible circuits may communicate electrical signals from the electrodes indicative of electrophysiological characteristics (and may be used for electrophysiology mapping, impedance/voltage detection), and/or communicate electrical signals indicative of temperature (where the one or more flex circuits include thermocouples). The flexible circuits 291_{A-D} may be coupled to the conductive tip 201 using known methods (e.g., adhesive, a re-flow process, etc.). In the present embodiment, the flexible circuits 291_{A-D} extend distally within trenches (which extent into an outer surface of the distal tip) which facilitate positioning of the electrodes 206₁₋₄ substantially flush with the outer surface of the distal tip (as further discussed in reference to FIGS. 4A-4C). As a result, the electrodes 206₁₋₄ facilitate a smooth, atraumatic transition between the conductive tip, flexible circuits, and conductive shell.

In some further embodiments, flexible circuits 291_{A-D} may include one or more interleaved conductive and non-conductive layers. One of the interior conductive layers of the flexible circuit may include thermocouples (which may be directly underneath the electrodes 206₁₋₄) to facilitate temperature sensing of tissue in contact with the flexible circuits. Placement of the one or more flex circuits into direct contact with tissue improves temperature sensing of the thermocouples therein (or thereon). Moreover, the thermocouples are not as susceptible to signal error associated with a flow of irrigant through the catheter shaft.

In yet other embodiments of the present disclosure, a single flexible circuit including the plurality of electrodes may extend circumferentially about the catheter shaft. The flexible circuitry may extend within a trench (which extends between conductive shell 202 and conductive tip 201) that facilitates positioning of the electrodes 206₁₋₄ substantially flush with an outer surface of the distal tip assembly 200. As a result, the inset placement of the electrodes 206₁₋₄ within the trench facilitates a smooth, atraumatic transition between the conductive shell, conductive tip and electrode faces.

With further reference to the embodiment of FIGS. 2A-C, the various electrical signals indicative of temperature and/or electrophysiology signals of the contacted tissue may be transmitted to a connector in a catheter handle via flex circuits 291_{A-D} extending a length of the catheter shaft 204. Alternatively, the electrical signals may be transmitted in conjunction with one or more additional flex circuit(s) joined to the flex circuits 291_{A-D} or via wires communicatively coupled to one or more of the flex circuits 291_{A-D} and extending proximally the remaining length of the catheter shaft to the catheter handle.

When the electrodes are placed into contact with tissue (e.g., myocardial tissue), the electrodes receive electrical signal information indicative of the electrophysiological characteristics of the tissue.

In various embodiments of the present disclosure, it may be necessary or desirable to isolate the electrodes 206₁₋₄ from a conductive tip 201 and conductive shell 202 of the distal tip assembly 200 to reduce RF-related interference received by the electrodes 206₁₋₄. Accordingly, various embodiments of the present disclosure are directed to distal tip assemblies where the flexible circuits 291_{A-D} may be electrically insulated from RF-related interference, but thermally conductive so as to facilitate near real-time temperature sensing of the target tissue and/or ablation electrode via one or more thermocouples integrated into the flexible circuits. In some specific embodiments, an electrically insulative material may at least partially circumscribe one or more of the electrodes 206₁₋₄ to prevent/limit RF-related signal interference from being received by the electrodes.

Conductive shell 202 and conductive tip 201, disclosed herein, may comprise for example platinum, a platinum iridium composition, or gold. The conductive shell may comprise one or more parts or components (including the conductive tip 201). As shown in FIGS. 2A and 2B, the conductive shell may comprise a hemispherical or nearly-hemispherical domed distal end (conductive tip 201) and a cylindrical body (conductive shell 202). In one embodiment, the wall thickness of the conductive shell is 0.002 inches, but alternative wall thicknesses may also be utilized. The conductive shell may be formed or manufactured by, for example, forging, machining, drawing, spinning, or coining. Also, the conductive shell may be constructed from molded ceramic that has, for example, sputtered platinum on its external surface. In another alternative embodiment, the conductive shell may be constructed from conductive ceramic material.

In various embodiments of the present disclosure the electrodes may be spot electrodes, or microelectrodes. The surface of these microelectrodes may be, for example, copper, gold-plated, Pt/Ir plated, or plated/coated with another material that provides superior electrograms and tissue impedance data, as well as superior durability. In various embodiments, the flexible circuits 291_{A-D} may include four electrodes each and one or more thermocouples, but the shape, size, and number of electrodes and/or thermocouples may vary from the embodiments shown, depending on specific design applications.

As further shown in FIGS. 2B and 2C, flexible circuits 291_{A-D} exit an internal cavity of distal tip assembly 200 via one or more apertures between conductive tip 201 and conductive shell 202. The flexible circuits then extend distally and radially inward within routed grooves to a longitudinal axis of the tip. The distal ends of each of the flex circuits may then be bonded together in the center of the tip with adhesive, with a stake 295, or by other possible means.

Alternative to the embodiment shown in FIGS. 2A-2C, the flex circuits 291_{A-D} may be routed through an aperture that extends through a longitudinal axis of the conductive tip 201. Each of the flex circuits 291_{A-D} may then extend proximally along trenches in the outer diameter of the conductive tip (or along an outer diameter of the conductive tip), and affixed thereto. In such an embodiment, radially extending apertures between the conductive tip 201 and conductive shell 202 (as shown in FIGS. 4A-4C) would not be required.

FIG. 2D is a perspective side view of a partial distal tip assembly 200′ of the distal tip assembly 200 of FIG. 2A, sans catheter shaft 204 to further illustrate an optical force sensing system 248.

The partial distal tip assembly 200′ includes a conductive tip 201 and conductive shell 202 at a distal portion 205. The conductive shell includes a member 207 and a plurality of irrigant apertures 208_{1-N}. The conductive shell 202 is coupled to a distal end of a manifold 215. The manifold 215 may be comprised of, for example, a stainless steel alloy, MP35N (a cobalt chrome alloy), titanium alloy, or a composition thereof. The member 207 facilitates deformation of the flex tip in response to contact with tissue; more specifically, the member 207 deforms to increase surface contact with target tissue. The increased tissue surface contact improves outcomes for various diagnostics and therapies (e.g., tissue ablation). After contact with target tissue is complete, the member 207 returns to an un-deformed state. A conductive tip 201 may be coupled to the member 207 of conductive shell 202 via an adhesive, weld, etc. The manifold 215, and an irrigation lumen 216 therein (as shown in FIG. 2C), extends through structural member 230, delivering irrigant from the irrigation lumen to a dispersion chamber within the conductive shell 202.

In various embodiments of the present disclosure, to limit the deformation of a structural member 230, distal tip assembly 200′ may transmit a portion of a force exerted on the ablation electrode through the manifold 215 (bypassing structural member 230). The manifold 215 transmits the force to a catheter shaft 204 that is coupled to a proximal end of the tip assembly 200 (as shown in FIGS. 2A-C).

Structural member 230 houses a plurality of fiber optic cables 240₁₋₃ that extend through grooves in an outer diameter of the structural member, for example groove 233₁. In the present embodiment, the structural member 230 is divided into a plurality of segments along a longitudinal axis. The segments are bridged by flexure portions 231₁₋₂, each flexure portion defining a neutral axis. Each of the neutral axes constitute a location within the respective flexure portions where the stress is zero when subjected to a pure bending moment in any direction.

In an optical force sensing system 248 of the present embodiment, fiber optic cables 240₁₋₃ may be disposed in grooves 233, respectively, such that the distal ends of the fiber optic cables terminate at the gaps of respective flexure portion 231₁₋₂. As shown in FIG. 2D, flexure portions 231₁₋₂ define a semi-circular segment that intercepts an inner diameter of structural member 230. The flexure portions 231₁₋₂ may be formed by one of the various ways available to an artisan, such as but not limited to sawing, laser cutting or electro-discharge machining (commonly referred to as EDM).

When an optical force sensing system consistent with the above is assembled, one or more fiber optic cables 240 are mechanically coupled to structural member 230 via grooves 233. In some embodiments, each of the fiber optic cables may be communicatively coupled to a Fabry-Perot strain sensor within one of the gaps which form the flexure portions 231₁₋₂. The Fabry-Perot strain sensor includes transmitting and reflecting elements on either side of the slots to define an interferometric gap. The free end of the transmitting element may be faced with a semi-reflecting surface, and the free end of the reflecting element may be faced with a semi-reflecting surface.

In some embodiments, structural member 230 may comprise a composition including a stainless steel alloy (or other metal alloy with characteristics including a high tensile strength, e.g., titanium), or platinum iridium (e.g., in a 90/10 ratio).

Further referring to the structural member 230, the structural member 230 is designed in such a way as to receive forces exerted on tip 201 and/or shell 202, and to absorb such force by deflecting and deforming in response thereto. Further, and as discussed in more detail above, the structural member 230 may be outfitted with a measurement device which facilitates measurement of the deflection/deformation of the deformable body which may be correlated with the force exerted on a distal portion 205 of the catheter 200′ and communicated with a clinician. Knowledge of a force exerted on the distal portion 205 of the catheter may be useful for a number of different cardiovascular operations; for example, during a myocardial tissue ablation therapy it is desirable to know a contact force exerted by the distal portion of the catheter on target tissue as tissue necrosis time is based on energy transferred between the catheter and tissue, and the extent of tissue contact.

In the various catheter tip assemblies disclosed herein, various electronic components in the catheter tip are necessary to facilitate desired functionality. As discussed in more detail above, the catheter tip may include, for example, one or more radio-frequency ablation electrodes, one or more electrodes, and/or a plurality of thermocouples. All of these electronic components must be communicatively coupled to catheter control circuitry at a proximal end of the catheter (as discussed above in reference to FIG. 1). Prior art ablation catheter systems utilize individual lead wires, extending the length of the catheter shaft, to facilitate communication between the various distal tip components and the catheter control circuitry. Aspects of the present disclosure are directed to reduced catheter assembly complexity by using one or more flexible circuits which extend at least a portion of the length of the catheter shaft, and communicatively couple the electronic components to the catheter control system.

FIG. 2E is a cross-sectional side view of a proximal portion 210 of the distal tip assembly 200 of FIG. 2A. The cross-sectional side view facilitates an understanding of irrigant flow through the proximal portion of the ablation catheter tip assembly. The irrigant flows from an irrigant source, through a catheter handle, and into a lumen of catheter shaft 204. The lumen delivers the irrigant to a distal end of the catheter shaft 204. Upon arriving at the distal end of the catheter shaft, the irrigant transitions into irrigant lumen 216 of manifold 215 via end cap 251. The manifold 215 delivers irrigant to a proximal end of conductive shell 202 (as shown in FIG. 2C), the irrigant is then circumferentially distributed through irrigant apertures 208_{1-N} in the conductive shell 202 via positive pressure.

A structural member 230 may be coupled at a distal end to a distal end of manifold 215, and at a proximal end to both the manifold 215 and an end cap 251. In some embodiments, the end cap may be made of platinum, titanium alloy, stainless steel alloy, MP35N (a cobalt chrome alloy), or a combination thereof. Once the distal tip assembly 200 is complete, the structural member 230 may be further coupled at a proximal end to a catheter shaft 204. The catheter shaft extending proximally to a catheter handle. The structural member 230 receives a force exerted on a distal portion 205 of the distal tip assembly 200 and absorbs such force by deflecting and deforming in response thereto. Further, and as discussed in more detail above, the structural member 230 may incorporate an optical force sensing system which facilitates measurement of the deflection/deformation of the deformable body. This deflection/deformation may then be correlated with the force (and in some cases the vector) exerted on a distal portion 205 of the distal tip assembly 200, and communicated to a clinician.

As shown in FIG. 2E, one or more flexible circuits 291_{A-B} are routed through an irrigant lumen 216 of manifold 215. Similarly, irrigant delivered to (and through) conductive shell 207 (as shown in FIG. 2D) flows through the irrigant lumen 216 of the manifold 215. First, however, the irrigant travels through end cap 251 via irrigant aperture 251_{C} into the irrigant lumen 216. The irrigant then flows along a length of the irrigant lumen and around the one or more flexible circuits into the conductive shell, before exiting through irrigant apertures 208_{1-N} and 208′₁₋₄ (as shown in FIGS. 2A-D).

As shown in FIG. 2E, one or more flexible circuits 291_{A-B} extend proximally through irrigant lumen 216 of manifold 215, through auxiliary apertures 251_{A,B} in end cap 251, and into an interior lumen of catheter shaft 204. The one or more flexible circuits 291_{A-B} may extend a length of the catheter shaft, or otherwise, may be communicatively coupled to another flexible circuit or a plurality of lead wires somewhere within the catheter shaft. In such embodiments, the one or more flexible circuits 291_{A-B} may include one or more connectors (or solder pads) at proximal ends of the respective circuits which facilitate electrical coupling to another flexible circuit or lead wires.

In various embodiments, the one or more flexible circuits 291_{A-B} are integrated into a single flexible circuit in the distal tip assembly 200, and include a plurality of thermocouples with are placed into (direct or indirect) thermal contact with the conductive shell 207 and/or patient tissue to conduct high-thermal sensitivity monitoring during an ablation therapy. In yet further embodiments, the one or more flexible circuits in the distal tip assembly includes one or more microelectrodes which facilitate electrophysiology monitoring of tissue in contact with the conductive shell and/or conductive tip 201. While not shown in the embodiments illustrated with reference to FIGS. 2A-2E, each of the one or more flexible circuits may further include a plurality of thermocouples positioned, for example, adjacent to the electrodes 206₁₋₄ and/or beneath the electrodes (e.g., on an inner substrate layer of a circuit board comprising one of the one or more flexible circuits).

While the embodiment of FIGS. 2A-E envision routing one or more flexible circuits 291_{A, B} at least partially through an irrigant lumen, various other possible routing paths are readily envisioned. For example, the flexible circuits may run proximally along an exterior of the catheter shaft 204, or within the catheter shaft (but outside of the irrigant lumen).

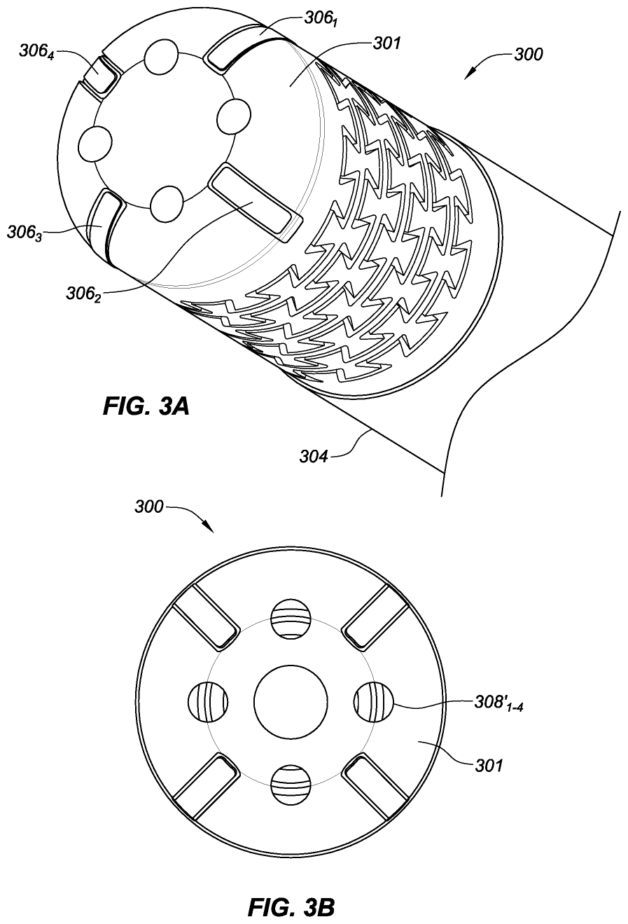

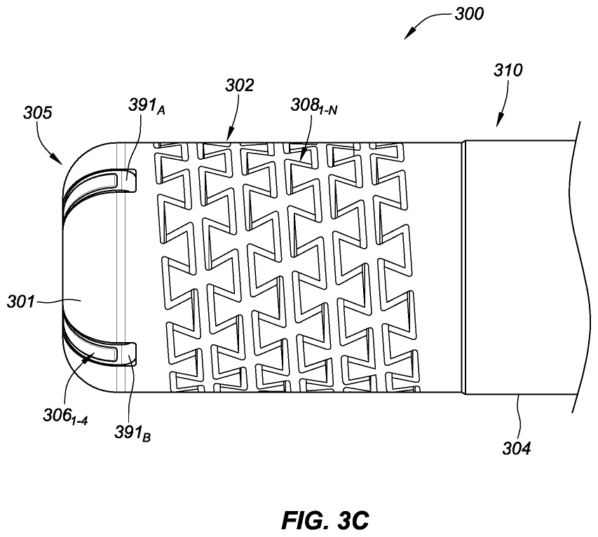

FIG. 3A is a perspective side view of an ablation catheter tip assembly 300, FIG. 3B is a front view of the ablation catheter tip assembly of FIG. 3A, and FIG. 3C is a side view of the ablation catheter tip assembly of FIG. 3A.

The ablation catheter tip assembly 300 includes a conductive tip 301 and a conductive shell 302 coupled to a distal end of a catheter shaft 304. In the present embodiment, the conductive shell and conductive tip are capable of receiving radio-frequency energy from a radio-frequency generator (e.g., RF Generator 40 as shown in FIG. 1), and transmitting that energy into myocardial tissue in contact with the conductive shell and/or conductive tip (or in close proximity thereto). The ablation catheter tip is irrigated and includes a plurality of irrigant apertures 308_{1-N} which extend through the conductive shell 302 and a plurality of distal irrigant apertures 308′₁₋₄ which extend through the conductive tip 301. The plurality of irrigant apertures deliver irrigant into contact with the targeted tissue for ablation therapy.

At a distal end 305 of the tip assembly 300, and in some embodiments at a juncture between the conductive shell 302 and the conductive tip 301, flexible circuits 391_{A-D} may extend from an interior cavity of the catheter and extend distally along a surface of the conductive tip. The flexible circuits 391_{A-D} may all be entirely separate flex circuits or be separate fingers of a common flex circuit. The flexible circuits 391_{A-D} include electrodes 306₁₋₄ (or be communicatively coupled thereto) which facilitate the detection of electrophysiological characteristics of tissue in contact with conductive tip 301 of the tip assembly 300. The flexible circuits 391_{A-D} may be coupled to the conductive tip 301 using known methods (e.g., adhesive, a re-flow process, etc.).

In the present embodiment, the flexible circuits 391_{A-D} extend distally within trenches (which extend into an outer surface of the distal tip) and which facilitate positioning sensing surfaces of the electrodes 306₁₋₄ substantially flush with the outer surface of the conductive tip (as further discussed in reference to FIGS. 4A-4C). As a result, the electrodes 306₁₋₄ facilitate a smooth, atraumatic transition between the conductive tip, flexible circuits, and conductive shell. In yet other embodiments, the electrodes on the flexible circuits may be positioned above a surface of the conductive tip to facilitate improved tissue contact. In further embodiments, the electrodes on the flexible circuits may be non-contact type electrodes and the trenches may be sufficiently deep to place a contact surface of the electrodes below the outer surface of the conductive tip.

As discussed in reference to FIGS. 2A-C, flexible circuits 391_{A-D} may include a plurality of thermocouples which may be positioned on a top layer of the circuit board or in a layer underneath the electrodes 306₁₋₄, among various other locations/configurations on the flexible circuit. For example, fingers of the flexible circuit including electrodes may extend outside of the catheter to facilitate direct electrical contact between a sensing surface of the electrodes and tissue, while additional fingers of the flexible circuit including thermocouples may be exclusively routed through an interior of the catheter and placed into direct contact with conductive tip 301 and/or conductive shell 302.

As further shown in FIGS. 3A-C, flexible circuits 391_{A-D} exit an internal cavity of distal tip assembly 300 via one or more apertures between conductive tip 301 and conductive shell 302. The flexible circuits then extend distally and radially inward within routed grooves, with the respective grooves and corresponding flexible circuits ending before intersecting at a longitudinal axis of the tip. Each of the flex circuits may be bonded within the grooves with adhesive, or by other coupling means.

FIG. 4A is a perspective side view of an ablation electrode 400, FIG. 4B is a back view of the ablation electrode of FIG. 4A, and FIG. 4C is a perspective back view of the ablation electrode of FIG. 4A. The ablation electrode 400 includes a conductive shell 402 and conductive tip 401 which are coupled to one another. In the present embodiment, the ablation electrode 400 is an irrigated ablation electrode including a plurality of irrigant apertures 408_{1-N} extending through the conductive shell 402 and distal irrigant apertures 408′₁₋₄ extending through the conductive tip (parallel to a longitudinal axis of the catheter). A proximal portion 410 of the conductive shell 402 may be configured for coupling to a catheter shaft and/or optical force measurement system.

To facilitate placement of electrodes and/or thermocouples on a distal portion 405 of the ablation electrode 400, aspects of the present disclosure are directed to facilitating routing of one or more flexible circuits out of an inner cavity of the ablation electrode and along an outer surface thereof. Radially extending access apertures 442₁₋₄ are circumferentially distributed about a longitudinal axis of the catheter shaft. Each of the apertures extending radially outward between the conductive shell 402 and conductive tip 401. In embodiments where the ablation electrode 400 is formed of a single body, the radially extending access apertures may extend through an outer surface of the ablation electrode at a location proximal the conductive tip and/or on the conductive tip itself.

Each of the radially extending access apertures 442₁₋₄ may be positioned adjacent a proximal end of a trench 441₁₋₄. Each of the respective trenches extend distally and radially inward along an outer surface of the conductive tip 401 toward a longitudinal axis of the catheter shaft. In the present embodiment, each of the trenches intersect at the longitudinal axis and an axial aperture 440 extends proximally through the conductive tip. The depth of the trench may be selected to facilitate flush placement of an electrode's sensing surface with an outer surface of the conductive tip.

During assembly of an ablation catheter tip in accordance with the present disclosure, flexible circuits may be routed from an interior cavity of the ablation electrode 400 and into trenches 441₁₋₄ of the conductive tip 401 via one or more of radially extending access apertures 442₁₋₄ and axial aperture 440. Each of the flexible circuits may then be coupled to the conductive tip 401 and the apertures 440 and 442 may be sealed with an adhesive.

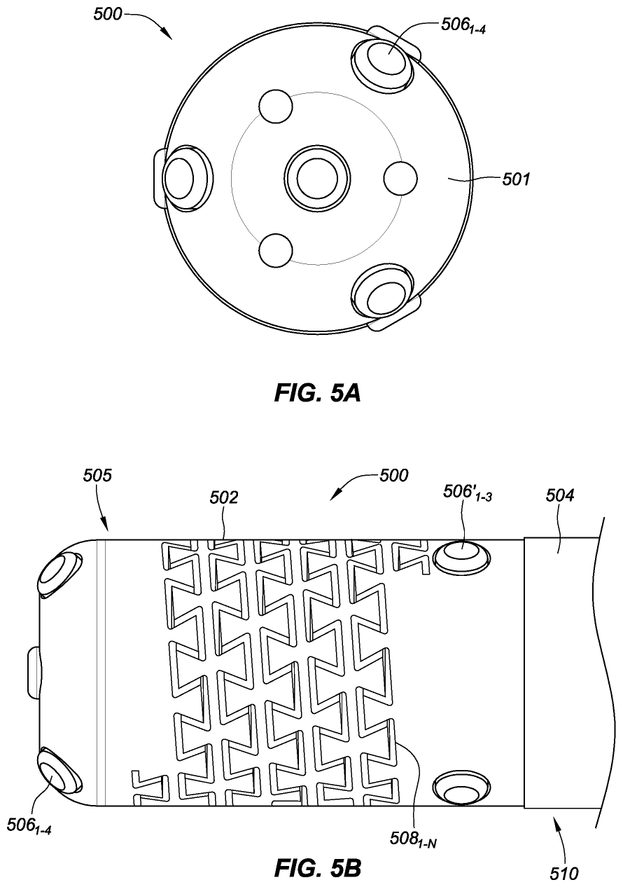

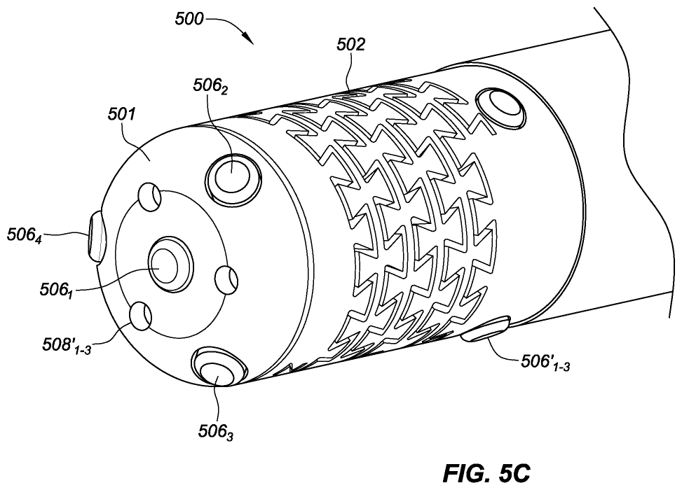

FIG. 5A is a front view of an ablation catheter tip assembly 500, FIG. 5B is a side view of the ablation catheter tip assembly of FIG. 5A, FIG. 5C is a perspective side view of the ablation catheter tip assembly of FIG. 5A, consistent with various embodiments of the present disclosure.

The ablation catheter tip assembly 500 includes a conductive tip 501 and a conductive shell 502 coupled to a distal end of a catheter shaft 504. In the present embodiment, the conductive shell and conductive tip are capable of receiving radio-frequency energy from a radio-frequency generator (e.g., RF Generator 40 as shown in FIG. 1), and transmitting that energy into myocardial tissue in contact with the conductive shell and/or conductive tip (or in close proximity thereto). In the present embodiment, the ablation catheter tip is irrigated and includes a plurality of irrigant apertures 508_{1-N} which extend through the conductive shell 502 and a plurality of distal irrigant apertures 508′₁₋₃ which extend through the conductive tip 501. The plurality of irrigant apertures deliver irrigant into contact with the targeted tissue for ablation therapy. While the present catheter embodiment is irrigated, aspects of the present disclosure may be readily applied to irrigated and non-irrigated catheters.

The tip assembly 500 further includes a plurality of apertures which facilitate the extension of distal electrodes 506₁₋₄ through the conductive tip 501 and proximal electrodes 506′₁₋₃ through conductive shell 502. In the present embodiment, the electrodes are microelectrodes which extend above a surface of the ablation electrode comprised of the conductive tip 501 and conductive shell 502. In various embodiments, the microelectrodes are communicatively coupled to one or more flexible circuits within an internal cavity of the ablation electrode. The electrodes 506₁₋₄ and 506′₁₋₃ facilitate the detection of electrophysiological characteristics of tissue in contact with the ablation electrode. The one or more flexible circuits may be coupled to the electrodes using known methods.

Placement of sensing surfaces of the electrodes above an exterior surface of the ablation electrode may facilitate enhanced conductivity between target tissue and the electrodes. In further embodiments, the electrodes may be non-contact type electrodes and may be recessed in relation to an outer surface of the ablation electrode. In yet further embodiments, a sensing surface of the electrode may be flush with an outer surface of the ablation electrode.

The one or more flexible circuits, positioned within an internal cavity of the ablation electrode 500, may include a plurality of thermocouples which may be positioned in thermally transmissive contact with the ablation electrode.

In the embodiment disclosed in reference to FIGS. 5A-C, the proximal and distal electrodes, 506′₁₋₃ and 506₁₋₄, respectively, are radially aligned with one another relative to a longitudinal axis of the catheter shaft. In yet other embodiments, the proximal and distal electrodes may be radially offset relative to one another. For example, where the distal and proximal electrodes each respectively include three electrodes circumferentially distributed about the outer surface of the ablation electrode, the proximal and distal electrodes may be radially offset by approximately 60°. Various other radial offsets are readily envisioned by a skilled artisan, such as to facilitate specific application sensing needs (e.g., conformance to a specific target structure—an ostium of a pulmonary vein).

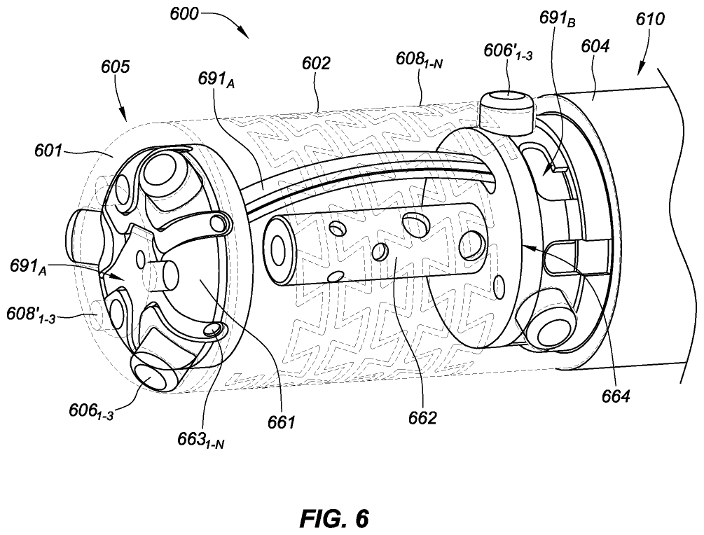

As discussed in more detail in relation to FIG. 6, one or more inserts within an internal cavity of ablation electrode may be used to position flexible circuits (including thermal couples) and the microelectrodes relative to the ablation electrode.

As further shown in the present embodiment, distal electrode 506₁ extends through an axial aperture in conductive tip 501, and is thereby aligned with the longitudinal axis of the catheter. The additional, distal electrodes 506₂₋₄ are positioned on a transition region between the conductive tip and the conductive shell. As a result, the distal electrodes are therefore oriented at an angle relative to the longitudinal axis of the catheter shaft to facilitate detection of electrophysiology characteristics of tissue in contact with the conductive tip 501 and/or the conductive shell 502. Proximal electrodes 506′₁₋₃ are aligned with and extend outward in a direction that is perpendicular to the longitudinal axis of the catheter shaft.

FIG. 6 is a perspective side view of an ablation catheter tip assembly 600, consistent with various embodiments of the present disclosure. A conductive shell 602 of the ablation catheter tip assembly is illustrated in transparency to facilitate discussion of the internal features of the assembly.

As shown in FIG. 6, flexible circuit 691_{A} extends distally from a catheter handle through catheter shaft 604, before transitioning into an irrigant lumen at an irrigant manifold (not shown). At a distal end of the irrigant manifold, the flexible circuit 691_{A} may be communicatively coupled to a second flexible circuit 691_{B} that extends radially outward and circumferentially wraps about an inner diameter of the conductive shell 602. This may be at a proximal end 610 of the conductive shell, and in close proximity to a coupling point between the conductive shell and the catheter shaft 604. The second flexible circuit 691_{B} may be positively positioned against the inner diameter of the conductive shell 602 via a proximal insert 664. Proximal electrodes 606′₁₋₃ are communicatively (and mechanically) coupled to the second flexible circuit, and may also be coupled to the proximal insert. The proximal electrodes may extend radially through apertures in the conductive shell 602 to facilitate direct contact between a sensing surface of the proximal electrodes and tissue.

In various embodiments of the present disclosure, the second flexible circuit 691_{B} may include one or more thermocouples which are placed into thermally transmissive contact with the conductive shell 602.

As shown in FIG. 6, the flexible circuit 691_{A} further extends through an internal cavity of conductive shell 602 to a distal portion 605 of the ablation catheter tip assembly 600. At the interface of the distal portion 605, the flexible circuit 691_{A} is coupled to a distal insert 661 which positively positions the flexible circuit, including a plurality of thermocouples 663_{1-N}, against an inner surface of the conductive tip 601. Distal electrodes 606₁₋₃ are communicatively (and mechanically) coupled to the flexible circuit, the distal electrodes may also be coupled to the distal insert 661. The distal electrodes 606₁₋₃ extend through apertures in the conductive tip 601, more specifically through a transition region between the tip and shell, to facilitate direct contact between a sensing surface of the proximal electrodes and tissue.

As shown in the present embodiment, the distal electrodes 606₁₋₃ are not mechanically coupled directly to the flexible circuit 691_{A}, but instead are coupled to the insert and communicatively coupled to the flexible circuit using lead wires and/or auxiliary flexible circuits, ZIFF-type connectors, among other connecting means readily known to a skilled artisan. In yet other embodiments, lead wires from each of the distal electrodes may be routed proximally to a catheter handle alongside flexible circuit 691_{A}.

The distal insert 661 may include trenches which further facilitate positive positioning of the flexible circuit 691_{A} relative to both the insert itself and an inner surface of conductive tip 601.

As in many embodiments of the present disclosure, ablation catheter tip assembly 600 is an irrigated catheter including a plurality of irrigant apertures 608_{1-N} which extend through the conductive shell 602 and a plurality of distal irrigant apertures 608′₁₋₃ which extend through the conductive tip 601. The plurality of irrigant apertures deliver irrigant into contact with the targeted tissue for ablation therapy. To further facilitate even distribution of irrigant to the plurality of irrigant apertures, an internal cavity of the ablation electrode may include an irrigant distributor 662. In yet further embodiments, the ablation catheter tip assembly 600 may be implemented as a non-irrigated catheter.

During assembly of the ablation catheter tip assembly 600, the proximal and distal flexible circuits may be wrapped around and secured to the respective inserts, by, for example using an adhesive backing on the flexible circuits, or adhesive placed within trenches on the inserts to which the flexible circuits mate. The distal insert is then inserted into the tip shell and secured in place via mechanical means (e.g., press fit, adhesive, held in place by a tip spring, etc.). The proximal insert may be press fit over an irrigation manifold, and the resulting assembly mechanically fixed in place (partially within the conductive shell), using for example a weld. The flexible circuit 691_{A} may then be routed proximally through channels in the proximal insert and through the catheter shaft to the catheter handle.

In various embodiments of the catheter tip assemblies disclosed herein, the catheter tip assemblies may also include a plurality of electrodes on a conductive shell thereof which facilitate electrophysiology mapping of tissue, such as myocardial tissue, in (near) contact with the shell. In more specific embodiments, the plurality of electrodes may be placed across the shell in such a manner as to facilitate Orientation Independent Algorithms which enhance electrophysiology mapping of the target tissue and is further disclosed in U.S. application Ser. No. 15/152,496, filed 11 May 2016, U.S. application Ser. No. 14/782,134, filed 7 May 2014, U.S. application Ser. No. 15/118,524, filed 25 Feb. 2015, U.S. application Ser. No. 15/118,522, filed 25 Feb. 2015, and U.S. application No. 62/485,875, filed 14 Apr. 2017, all of which are now pending, and are incorporated by reference as though fully disclosed herein.

While various embodiments of the present disclosure, including FIGS. 2-4, are directed to ablation catheter tips including two rings of 3 radially-disposed thermal sensors, the invention is not limited to such an seven-sensor configuration (or eight-sensor configuration where an additional electrode is placed along a longitudinal axis of the distal tip). Various other configurations are readily envisioned.

It is to be understood that while an irrigated ablation catheter tip is illustrated in various embodiments of the present disclosure, the design of the structural assembly (including structural member, manifold, and end cap) is modular and may facilitate the fitting of various catheter tips (e.g., rigid, flex, and other advanced irrigation tips).

Applicant further envisions utilizing catheters comprising various segmented tip designs with the ablation catheter system described above. Example tip configurations are disclosed in U.S. patent application No. 61/896,304, filed 28 Oct. 2013, and in related international patent application no. PCT/US2014/062562, filed 28 Oct. 2014 and published 7 May 2015 in English as international publication no. WO 2015/065966 A2, both of which are hereby incorporated by reference as though fully set forth herein.

Although several embodiments have been described above with a certain degree of particularity, those skilled in the art could make numerous alterations to the disclosed embodiments without departing from the present disclosure. It is intended that all matter contained in the above description or shown in the accompanying drawings shall be interpreted as illustrative only and not limiting. Changes in detail or structure may be made without departing from the present teachings. The foregoing description and following claims are intended to cover all such modifications and variations.

Various embodiments are described herein of various apparatuses, systems, and methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” “an embodiment,” or the like, means that a particular feature, structure, or characteristic described in connection with the embodiment is included in at least one embodiment. Thus, appearances of the phrases “in various embodiments,” “in some embodiments,” “in one embodiment,” “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A high-thermal-sensitivity ablation catheter tip, the tip comprising:
an ablation electrode configured and arranged to deliver an ablation therapy to tissue in contact with or in close proximity thereto; and
a flexible electronic circuit coupled to an external surface of the ablation electrode, the flexible electronic circuit including one or more electrodes configured and arranged to sense electrophysiology characteristics of the tissue in contact with or in close proximity to the ablation electrode.

2. The high-thermal-sensitivity ablation catheter tip of claim 1, wherein the ablation electrode includes a trench extending into the external surface, the flexible electronic circuit coupled within the trench, and the trench aligns a sensing surface of the one or more electrodes flush with the external surface of the ablation electrode.

3. The high-thermal-sensitivity ablation catheter tip of claim 2, wherein the flexible electronic circuit further includes one or more thermocouples, each of the thermocouples positioned on an intermediate layer of the flexible electronic circuit beneath one of the respective electrodes.

4. The high-thermal-sensitivity ablation catheter tip of claim 2, wherein the ablation electrode includes a radially extending access aperture adjacent the trench, the flexible electronic circuit is routed from an inner cavity of the ablation electrode and into the trench on the external surface of the ablation electrode via the radially extending access aperture.

5. The high-thermal-sensitivity ablation catheter tip of claim 4, wherein the ablation electrode includes additional trenches extending into the external surface of the ablation electrode and additional radially extending access apertures adjacent the additional trenches;
wherein the flexible electronic circuit includes a body member and a plurality of fingers which extend from the body member, the plurality of fingers extend through the radially extending access apertures in the ablation electrode and are routed along respective additional trenches; and
wherein each of the plurality of fingers include one or more electrodes, and the fingers are coupled within the respective additional trenches.

6. The high-thermal-sensitivity ablation catheter tip of claim 5, wherein the plurality of fingers of the flexible electronic circuit are coupled to the additional trenches, and the additional trenches align the sensing surfaces of the electrodes flush with the external surface of the ablation electrode; and
wherein the additional trenches are located on an ablation tip of the of the ablation electrode, and extend distally and radially inward along the external surface of the ablation electrode from respective radially extending access apertures.

7. The high-thermal-sensitivity ablation catheter tip of claim 2, wherein the trench is located on an ablation tip of the ablation electrode and extends distally and radially inward along the external surface of the ablation electrode.

8. The high-thermal-sensitivity ablation catheter tip of claim 1, wherein the ablation electrode includes a conductive tip and a conductive shell, the conductive shell including irrigant apertures extending therethrough, the irrigant apertures configured and arranged for irrigant distribution circumferentially about the conductive shell;
wherein the high-thermal-sensitivity ablation catheter tip further includes a manifold coupled to a proximal end of the conductive shell, the manifold including an irrigation lumen extending through a longitudinal axis, the irrigation lumen configured and arranged to deliver irrigant into an inner cavity of the conductive shell; and
wherein the flexible electronic circuit extends through at least a portion of the irrigation lumen.

9. The high-thermal-sensitivity ablation catheter tip of claim 1, further including
a second flexible electronic circuit circumferentially coupled to an inner surface of the ablation electrode, the second flexible electronic circuit including a second plurality of electrodes distributed along a length of the second flexible circuit; and
wherein the second plurality of electrodes extend radially outward through apertures in the ablation electrode.

10. The high-thermal-sensitivity ablation catheter tip of claim 1, wherein the flexible electronic circuit further includes one or more thermocouples; and
the high-thermal-sensitivity ablation catheter tip further includes a distal insert that is configured and arranged to position the flexible electronic circuit within an inner cavity of the ablation electrode and to position the one or more thermocouples into thermally transmissive contact with the ablation electrode.

11. The high-thermal-sensitivity ablation catheter tip of claim 1, wherein the one or more electrodes are electrically isolated from the ablation electrode.

12. The high-thermal-sensitivity ablation catheter tip of claim 1, wherein the ablation electrode includes a conductive tip, a conductive shell, and a transition region therebetween; and
wherein the one or more electrodes of the flexible electronic circuit are positioned within the transition region.

13. The high-thermal-sensitivity ablation catheter tip of claim 12, wherein the ablation electrode further includes distal irrigant apertures which extend through the conductive tip and are circumferentially interleaved between the one or more electrodes.

14. A high-thermal-sensitivity ablation catheter tip, the tip comprising:
an ablation electrode configured and arranged to deliver an ablation therapy to tissue in contact with or in close proximity thereto, the ablation electrode including a conductive tip, a conductive shell, and a transition region between the conductive tip and the conductive shell;
a distal flexible electronic circuit including a first plurality of electrodes which extend through apertures in a transition region of the ablation electrode; and
a proximal flexible electronic circuit including a second plurality of electrodes which extend through apertures at a proximal end of the conductive shell.

15. The high-thermal-sensitivity ablation catheter tip of claim 14, wherein the distal flexible electronic circuit further includes an additional electrode extending through the conductive tip and positioned along a longitudinal axis of the catheter tip.

16. The high-thermal-sensitivity ablation catheter tip of claim 14, further including a plurality of thermocouples communicatively coupled to the distal flexible electronic circuit, and a distal tip insert configured and arranged to position the distal flexible electronic circuit within an inner cavity of the ablation electrode and to position the plurality of thermocouples into thermally transmissive contact with the ablation electrode.

17. The high-thermal-sensitivity ablation catheter tip of claim 14, wherein the distal and proximal flexible electronic circuits are communicatively coupled to one another.

18. The high-thermal-sensitivity ablation catheter tip of claim 14, wherein the first and second plurality of electrodes are configured in two circumferential rings about the ablation electrode, where the electrodes of the first circumferential ring are radially offset by approximately 60° relative to the electrodes of the second circumferential ring.

19. The high-thermal-sensitivity ablation catheter tip of claim 14, wherein the conductive shell includes irrigant apertures extending therethrough, the irrigant apertures configured and arranged for irrigant distribution;
wherein the high-thermal-sensitivity ablation catheter tip further includes a manifold coupled to a proximal end of the ablation electrode, the manifold including an irrigation lumen extending through a longitudinal axis of the manifold, the irrigation lumen configured and arranged to deliver irrigant to the irrigant apertures; and
wherein at least one of the distal and proximal flexible electronic circuits extend through at least a portion of the irrigation lumen.
